(19) United States
(12) Patent Application Publication
(10) Pub. No.: US 2006/0247141 A1
(43) Pub. Date: Nov. 2, 2006

(54) Copolymerizable polyalkylene glycol macromonomers, and the preparation and use thereof

(75) Inventor: Martin Glos, Essen (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant Produkte (Deutschland) Gmbh)

(21) Appl. No.: 11/412,892

(22) Filed: Apr. 27, 2006

(30) Foreign Application Priority Data
Apr. 30, 2005 (DE) 102005020265.9
Jun. 2, 2005 (DE) 102005025269.9

(51) Int. Cl. A61K 8/18

(52) U.S. Cl. 510/101; 512/13

(57) ABSTRACT

The invention relates to compounds of the formulae 1 and 2 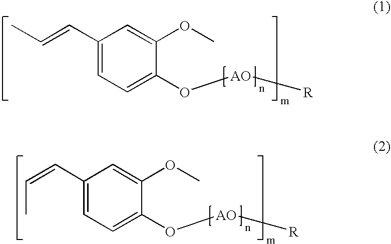 in which
A is C₂- to C₄-alkylene, n is an integer from 4 to 900, m is an integer from 1 to 50, and R is hydrogen, an m-valent acid group, or an m-valent organic radical having 1 to 200 carbon atoms which, apart from carbon and hydrogen, can also contain heteroatoms such as, for example, oxygen, nitrogen, sulfur or phosphorus, and to the use thereof as polymerizable emulsifiers.

The present invention relates to the synthesis of isoeugenol polyalkylene glycols, and to the use thereof, for example, as reactive emulsifiers or hydrolysis-stable macromonomers for diverse applications in the field of polymer synthesis or as reactive intermediates for syntheses or as fragrances or aromas.

Polyalkylene glycols are generally prepared by anionic, ring-opening polymerization of epoxides (such as, for example, ethylene oxide and propylene oxide) with alcohols as initiators (so-called starter alcohols), according to reaction equation 1. Catalysts which can be used here are catalytically effective amounts of alkali metal or alkaline earth metal hydroxides or the corresponding alkali metal alkoxides. Thus, for example, if R′—OH=methanol, then α-methoxy-Ω-hydroxypolyalkylene glycols are formed, and if R′—OH=butanol, then α-butoxy-Ω-hydroxypolyalkylene glycols are formed accordingly: 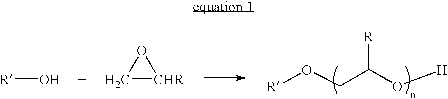
Polyalkylene glycol macromonomers, i.e. polyalkylene glycols which, in addition to the polyether chain, contain a terminal, reactive, copolymerizable double bond, are of interest for the preparation of comb polymers with polyalkylene glycol side groups for a large number of applications such as, for example, as reactive copolymerizable surfactants or, for example, as comonomer building blocks for the synthesis of polymeric dispersants. For preparing these polyalkylene glycol macromonomers with terminal double bond, there are in principle two options.

Starting from a polyalkylene glycol (e.g. methyl polyglycol), a terminal double bond can be introduced through reaction at the terminal hydroxy group, for example by esterification with methacrylic acid or other unsaturated acids (Gramain, Polymer Commun 1986, 27, 16 ff). A disadvantage here is the fact that this is an at least two-stage synthesis (1st stage: preparation of the polyalkylene glycol, 2nd stage:

esterification) and the polyglycol side chain which forms during the subsequent (co)polymerization of the macromonomer is bonded to the main chain via an ester group, and is therefore not fixed to the main chain in a hydrolysis-stable form.

Alternatively, starting from an olefinically unsaturated alcohol R′—OH according to equation 1, the polyalkylene glycol with terminal double bond can be synthesized directly. If R′—OH=allyl alcohol, then, in a single-stage reaction according to equation 1, α-allyloxy-Ω-hydroxypolyalkylene glycols are formed. If required, the α-1-allyloxy-Ω-hydroxypolyalkylene glycol which is formed primarily can also be converted into the corresponding α-1-allyloxy-Ω-alkoxypolyalkylene glycols by esterification with alkyl chlorides as described in DE-A-41 38 166.

In contrast to the above-described synthesis of polyalkylene glycol macromonomers by esterification, the polyether side chains of the resulting comb polymers in this process are linked to the polymer backbone via an ether compound and are thus largely hydrolysis-stable.

It was an object of the present invention to find cost-effective and storage-stable polyalkylene glycol macromonomers which, following copolymerization with other unsaturated comonomers, have a hydrolysis-stable chemical bonding of the polyether side chain in the resulting copolymer.

It has now been found that isoeugenol polyalkylene glycols satisfy these requirements and are obtainable by a simple route. They are suitable for copolymerizing with a large number of monomers, and in so doing of forming hydrolysis-stable copolymers (comb polymers) with polyalkylene glycol side chains.

The invention thus provides compounds of the formulae 1 and 2 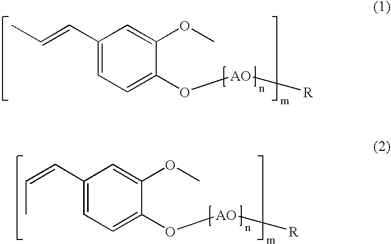
in which
A is C₂- to C₄-alkylene,
n is an integer from 4 to 900,
m is an integer from 1 to 50, and
R is hydrogen, an m-valent acid group, or an m-valent organic radical having 1 to 200 carbon atoms which, apart from carbon and hydrogen, can also contain heteroatoms such as, for example, oxygen, nitrogen, sulfur or phosphorus.

In the formulae 1 and 2, the trans form and the cis form of the isoeugenol derivatives according to the invention are shown. In the text below, only the trans form is depicted, although the cis form is likewise in accordance with the invention. The compounds of the formula 1 and 2 according to the invention are also referred to below as isoeugenol polyalkylene glycols.

The invention further provides the use of compounds of the formulae 1 and/or 2 as monomer in polymerization reactions.

The invention further provides the use of compounds of the formulae 1 and/or 2 as polymerizable emulsifiers.

The invention further provides a process for the preparation of an emulsified copolymer by adding a compound of the formula I and/or II to the olefinically unsaturated compounds capable of free-radical polymerization, and polymerizing the resulting mixture in aqueous phase.

The invention further provides copolymers and their dispersions comprising monomers of the formulae 1 and/or 2 and at least one further olefinically unsaturated monomer.

A is preferably ethylene radicals or mixtures of ethylene and propylene radicals.

m is preferably an integer from 1 to 10.

In the alkoxy chain represented by (A—O)ₙ, A is preferably an ethylene or propylene radical, in particular an ethylene radical. The total number of alkoxy units is preferably between 5 and 300, in particular between 8 and 200. The alkoxy chain may be a block polymer chain which has alternating blocks of different alkoxy units, preferably ethoxy and propoxy units. It may also be a chain with a random sequence of alkoxy units or a homopolymer.

In a preferred embodiment, —(A—O)ₘ— is an alkoxy chain of the formula 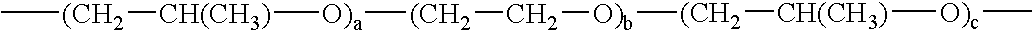
in which
a is a number from 0 to 300, preferably 0 to 80
b is a number from 5 to 300, preferably 5 to 200
c is a number from 0 to 300, preferably 0 to 80.

In a further preferred embodiment, —(A—O)ₙ— is an ethoxy radical having 8 to 240 ethoxy units.

A common aspect of all of the embodiments is that preferably at least 50 mol % of the radicals (A—O) are ethoxy radicals, in particular 60 to 100 mol % are ethoxy radicals.

R is an m-valent acid group or hydrogen or a saturated or unsaturated, cyclic or acyclic organic radical having 1 to 200 carbon atoms which, apart from carbon and hydrogen, can also contain heteroatoms such as, for example, oxygen, nitrogen, sulfur or phosphorus. If R is an acid group, then it may either be a monomeric or a polymeric acid group or a mono- or polyfunctional unit (m-functional). If R is hydrogen, then m is 1.

Examples of inorganic acids from which the acid group R can be derived are sulfuric acid and phosphoric acid. If sulfuric acid is used, then the compounds of the formula 1 and/or 2 can either be monoesters or diesters of sulfuric acid, i.e. m is 1 or 2. If phosphoric acid is used, then the compounds of the formulae 1 and/or 2 can either be monoesters, diesters or triesters of phosphoric acid, i.e. m is 1, 2 or 3.

In a preferred embodiment, the organic acids from which R can be derived are mono-, di-, tri- or polyvalent carboxylic acids, i.e. compounds which contain 1, 2, 3 or more carboxyl groups and which, moreover, can also have at least one sulfur- or phosphorus-containing functional group. Particular preference is given to sulfur-containing functional groups, specifically sulfonate groups. Preferred sulfonic acids/sulfonates may be aliphatic or aromatic compounds. Preferred sulfonic acids/sulfonates contain 2 or 3 carboxyl groups, and including the carboxyl groups, 3 to 6 carbon atoms. A particularly preferred sulfonic acid is sulfosuccinic acid.

In a preferred embodiment, the sulfonic acids and carboxylic acids are aromatic or aliphatic compounds which carry one or more acid functions.

The carboxylic acids may be unsaturated carboxylic acids, such as, for example, acrylic acid, methacrylic acid or maleic acid.

Preferred isoeugenol alkoxylates in which R is not hydrogen can, for example, have the following structures of the formulae 3 to 10, 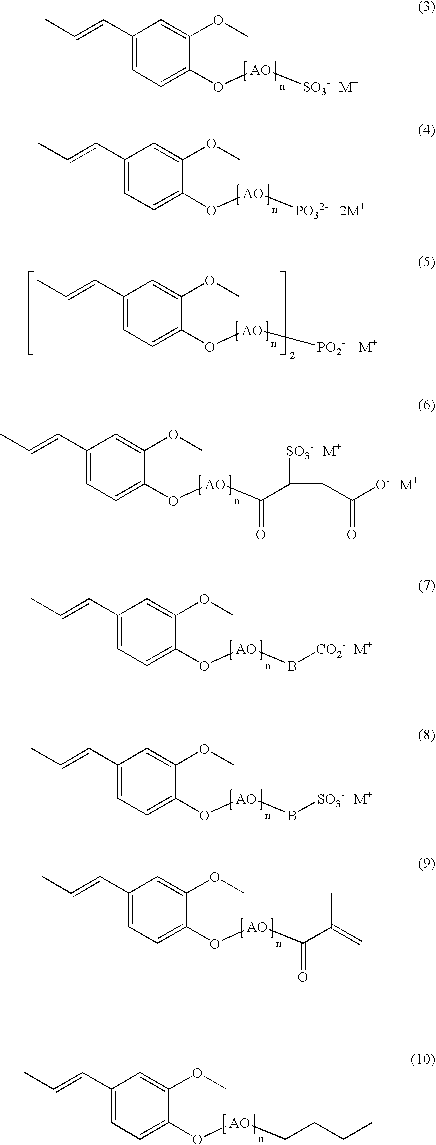
in which B is an aliphatic or aromatic group having 1 to 50 carbon atoms, which may also contain heteroatoms, and M⁺ is
a monovalent metal ion, such as, for example, an alkali metal ion,
an ammonium ion, such as, for example, NH₄⁺ or mono-, di-, tri- and/or tetraalkylammonium ions, where the alkyl substituents of these ammonium ions can, independently of one another, be C₁- to C₂₂-alkyl radicals, which may optionally be occupied by up to 30 C₂- to C₁₀-hydroxyalkyl groups,
H⁺, or
equivalents of di-, tri- or polyvalent metal ions, such as, for example, Ca²⁺ or Al³⁺.

The compounds according to the invention can be prepared by reacting the parent alkoxylates with suitable acids or corresponding acid derivatives. Sulfate esters are preferably prepared using amidosulfonic acid. The ammonium salts obtained thereby can be converted into the corresponding alkali metal salts by reaction with alkali metal hydroxides. For the preparation of phosphoric esters, phosphoric acid can be used. The methacrylic esters can be prepared by direct esterification with methacrylic acid or transesterification with, for example, methyl methacrylate. Organic acids can also be reacted in the form of their anhydrides with the parent alkoxylates. Functional groups can also be inserted following the preparation of the ester of the nonfunctionalized acid. For example, sulfosuccinic esters can be prepared by preparing the corresponding maleic esters and subsequent sulfonation, for example with pyrosulfites.

The sulfonic acids, carboxylic acids and phosphonic acids can also be prepared by reacting the parent alkoxylates with the corresponding halides or cyclic esters of the sulfonic or carboxylic acids.

The corresponding alkyl derivatives can be prepared by reacting the parent alkoxylates in a Williamson ether synthesis under alkaline conditions with alkyl halides, such as, for example, butyl chloride.

The compounds according to the invention are, with the exception of the corresponding ester of polymerizable acids such as, for example, according to formula 9, themselves not homopolymerizable since the propenyl-styryl group, in contrast to acrylic or methacrylic ester groups, is not in the position for homopolymerization (cf. B. Vollmert, Grundriss der Makromolekularen Chemie [Outline of Macromolecular Chemistry] volume I p. 55). The macromonomers according to the invention are thus storage-stable. They can, however, be induced to polymerize with further olefinically unsaturated compounds. This can take place in accordance with the processes known in the art (for example bulk polymerization, solution polymerization, emulsion polymerization). Through this, the resulting polymers can be produced, for example, as polymer dispersion, polymer solution or as solid polymer.

In the various polymerization methods, the radical starters known according to the prior art can be used.

Likewise, to control the molecular weight and to influence the polymer structure, crosslinking monomers and/or regulators can be used.

Free-radically initiated copolymerization of the polymerizable macromonomers with olefinically unsaturated comonomers produces the copolymers according to the invention. Comonomers which can be used are all olefinically unsaturated monomers whose reaction parameters allow a copolymerization with the compounds according to the invention in the particular reaction media.

These are, for example, open-chain N-vinylamides, preferably N-vinylformamide (VIFA), N-vinylmethylformamide, N-vinylmethylacetamide (VIMA) and N-vinylacetamide; cyclic N-vinylamides (N-vinyl lactams) with a ring size of from 3 to 9, preferably N-vinylpyrrolidone (NVP) and N-vinylcaprolactam; amides of acrylic acid and methacrylic acid, preferably acrylamide, methacrylamide, N,N-dimethylacrylamide, N,N-diethylacrylamide and N,N-diisopropylacrylamide; alkoxylated acryl- and methacrylamides, preferably hydroxyethyl methacrylate, hydroxymethylmethacrylamide, hydroxyethylmethacrylamide, hydroxypropylmethacrylamide and mono[2-(methacryloyloxy)ethyl] succinate;

N,N-dimethylaminomethacrylate; diethylaminomethyl methacrylate; acryl- and methacrylamidoglycolic acid; 2- and 4-vinylpyridine; vinyl acetate; glycidyl methacrylate; styrene; acrylonitrile.

Unsaturated amine derivatives, such as, for example, diallylamine, diallyldimethylammonium chloride or trimethyl-2-methacryloylethylammonium chloride.

Olefins, such as ethylene, propene and butenes, pentene, 1,3-butadiene and chlorbprene, vinyl halides, such as vinyl chloride, vinylidene chloride and vinylidene fluoride.

Alkoxylated allyl, vinyl compounds and vinyl ethers Olefinically unsaturated carboxylic esters based on a very wide variety of alcohols and unsaturated carboxylic acids, such as, for example, ethyl acrylate, n-butyl acrylate, butyl acrylate, hexyl acrylate, 2-ethylhexyl acrylate, hydroxyethyl acrylate, stearyl acrylate; lauryl methacrylate, dibutyl maleate, methyl polyglycol-1000 methacrylate, nonylphenol polyalkylene glycol methacrylates, tributylphenol polyalkylene glycol methacrylates etc.

In addition, one or more unsaturated carboxylic acids or salts thereof can be polymerized into the copolymer. Of particular preference are acrylic acid, methacrylic acid, styrenesulfonic acid, maleic acid, fumaric acid, crotonic acid, itaconic acid and senecioic acid.

Olefinically unsaturated sulfonic acids and phosphonic acids, such as acrylamidomethylpropanesulfonic acid, methallylsulfonic acid or vinylphosphonic acid derivatives can also likewise be used.

Preferred counterions of these acids are Li⁺, Na⁺, K⁺, Mg⁺⁺, Ca⁺⁺, Al⁺⁺⁺, NH₄⁺, monoalkylammonium, dialkylammonium, trialkylammonium and/or tetraalkylammonium radicals, where the alkyl substituents of the amines may, independently of one another, be (C₁-C₂₂)-alkyl radicals, which may optionally be occupied by up to 3 (C₂-C₁₀)-hydroxyalkyl groups. In addition, it is also possible to use mono- to triethoxylated ammonium compounds with varying degree of ethoxylation. The degree of neutralization of the carboxylic acids can be between 0 and 100%.

The copolymers according to the invention can optionally comprise comonomers with more than one polymerizable unit. Comonomers with more than one polymerizable unit lead to crosslinking of the structures according to the invention. In a corresponding embodiment, the copolymers comprise comonomers with at least two polymerizable vinyl groups.

Preferred crosslinkers are methylenebisacrylamide;

methylenebismethacrylamide; esters of unsaturated mono- and polycarboxylic acids with polyols, preferably diacrylates and triacrylates or -methacrylates (e.g.: PEG-400 dimethacrylate), particularly preferably butanediol and ethylene glycol diacrylate or methacrylate, trimethylolpropane triacrylate (TMPTA) and allyl compounds, preferably allyl (meth)acrylate, triallyl cyanurate, diallyl maleate, polyallyl esters, tetraallyloxyethane, triallylamine, tetraallylethylenediamine; allyl esters of phosphoric acid; and/or vinylphosphonic acid derivatives. A particularly preferred crosslinker is trimethylolpropane triacrylate (TMPTA) and trimethylolpropane trimethacrylate (TMPTMA).

Mixtures of vinylically monounsaturated comonomers with polyunsaturated comonomers (crosslinkers) are likewise in accordance with the invention.

The compounds according to the invention can be used as reactive emulsifiers either on their own or else in combination with other already known cationic, anionic and nonionic emulsifiers of the prior art.

In general, the compounds according to the invention are used as emulsifiers or comonomers in amounts of from 0.1 to 99% by weight, preferably 0.2 to 95% by weight, in particular 0.4 to 90% by weight, based on the weight of the olefinically unsaturated monomers used for the preparation of the polymers.

The isoeugenol polyalkylene glycols according to the invention can be prepared directly in one step by reacting eugenol or isoeugenol with ethylene oxide, propylene oxide or other epoxides under suitable reaction conditions. In this connection, in one step means without isolating an intermediate and is decisive for the economic feasibility of the process.

The examples below illustrate the invention in more detail.

### EXAMPLE 1

In a pressure reactor, 150 g of eugenol were admixed with 0.87 g of sodium methoxide dissolved in methanol and the mixture was then stirred under reduced pressure at 70° C. for 3 hours to remove methanol. Then, at a temperature of 160° C., 402 g of ethylene oxide were metered in slowly so that the pressure did not exceed 6 bar. The resulting polyether was neutralized with acetic acid to a pH of 7 and analyzed by means of ¹H NMR. In the NMR spectrum, signals of an isoeugenol ethoxylate with 10 ethylene glycol units per aromatic radical were found.

### EXAMPLE 2

In a pressure reactor, 200 g of isoeugenol were admixed with 1.2 g of sodium methoxide dissolved in methanol and the mixture was then stirred under reduced pressure at 70° C. for 3 hours to remove methanol. Then, at a temperature of 160° C., 537 g of ethylene oxide were metered in slowly so that the pressure did not exceed 6 bar. The resulting polyether was neutralized with lactic acid to a pH of 7 and analyzed by means of ¹H NMR. In the NMR spectrum, signals of an isoeugenol ethoxylate with 10 ethylene glycol units per aromatic radical were found.

### EXAMPLE 3

In a pressure reactor, 300 g of eugenol were admixed with 0.79 g of sodium methoxide dissolved in methanol and the mixture was then stirred under reduced pressure at 70° C. for 3 hours to remove methanol. Then, at a temperature of 160° C., 402 g of ethylene oxide were metered in slowly so that the pressure did not exceed 6 bar. The resulting polyether was neutralized with lactic acid to a pH of 7 and analyzed by means of ¹H NMR. In the NMR spectrum, signals of an isoeugenol ethoxylate with 5 ethylene glycol units per aromatic radical were found.

### EXAMPLE 4

In a pressure reactor, 160 g of eugenol were admixed with 1.48 g of sodium methoxide dissolved in methanol and the mixture was then stirred under reduced pressure at 70° C. for 3 hours to remove methanol. Then, at a temperature of 160° C., 860 g of ethylene oxide were metered in slowly so that the pressure did not exceed 6 bar. The resulting polyether was neutralized with lactic acid to a pH of 7 and analyzed by means of ¹H NMR. In the NMR spectrum, signals of an isoeugenol ethoxylate with 20 ethylene glycol units per aromatic radical were found.

### EXAMPLE 5

In a pressure reactor, 80 g of eugenol were admixed with 1.48 g of sodium methoxide dissolved in methanol and the mixture was then stirred under reduced pressure at 70° C. for 3 hours to remove methanol. Then, at a temperature of 160° C., 860 g of ethylene oxide were metered in slowly so that the pressure did not exceed 6 bar. The resulting polyether was neutralized with lactic acid to a pH of 7 and analyzed by means of ¹H NMR. In the NMR spectrum, signals of an isoeugenol ethoxylate with 40 ethylene glycol units per aromatic radical were found.

### EXAMPLE 6

In a pressure reactor, 200 g of eugenol were admixed with 2.6 g of sodium methoxide dissolved in methanol and the mixture was then stirred under reduced pressure at 70° C. for 3 hours to remove methanol. Then, at a temperature of 140° C., 354 g of propylene oxide were metered in slowly so that the pressure did not exceed 4 bar. The resulting polyether was neutralized with isononanoic acid to a pH of 7 and analyzed by means of ¹H NMR. In the NMR spectrum, signals of an isoeugenol propoxylate with 5 propylene glycol units per aromatic radical were found.

### EXAMPLE 7

In a pressure reactor, at a temperature of 160° C., 194 g of ethylene oxide were slowly metered into 200 g of the unneutralized product from Example 6 so that the pressure did not exceed 6 bar. The resulting polyether was neutralized with lactic acid to a pH of 7 and analyzed by means of ¹H NMR. In the NMR spectrum, signals of an isoeugenol alkoxylate with 5 propylene glycol units and 10 ethylene glycol units per aromatic radical were found.

### EXAMPLE 8

In a glass reactor, 140 g of the isoeugenol ethoxylate prepared in Example 4 were admixed, in a nitrogen atmosphere and with stirring, with 49 g of methyl methacrylate, 0.9 g of butylhydroxytoluene, 0.5 g of p-methoxyphenol and 1.4 g of dibutyltin oxide. The mixture was heated for 14 hours at 100-130° C., and the distillate which formed during this time was distilled off. Using NMR spectroscopy, a degree of conversion of >95% to the corresponding methacrylic ester was ascertained.

### EXAMPLE 9

In a glass reactor, 100 g of the isoeugenol ethoxylate prepared in Example 1 were admixed, with stirring, with 0.5 g of urea and 16 g of amido sulfonic acid and heated for 4 hours at 100° C. The pH was then adjusted to 6.5-7.5 with 0.25 g of 50% strength sodium hydroxide solution. By means of NMR spectroscopy, a degree of conversion of >95% to the corresponding sulfuric ester ammonium salt was ascertained.

### EXAMPLE 10

440 g of an alcohol which was prepared according to Example 1 was admixed with 85 g of polyphosphoric acid at 70° C. and stirred at 70° C. for 2 h. After a further 2 h at 100° C., 22 g of water were added at 90° C. and the mixture was stirred for a further 2 h. A product was obtained which was a mixture of 87% by weight of phosphoric monoester and 10% by weight of phosphoric diester, remainder water. The product comprised no phosphoric triester.

### EXAMPLE 11

300 g of an alcohol which was prepared according to Example 1 was added under nitrogen to 49 g of maleic anhydride at 70° C. The mixture was then heated at 90° C. for 8 h, during which the water which formed was distilled off. The product obtained was added to a mixture of 26 g of sodium pyrosulfite, 20 g of NaOH and 390 g of dist. water and heated at 80° C. for 5 h. This gave a solution of the corresponding sulfosuccinate with a content of 50% by weight.

1. A compound of the formula 1 or 2 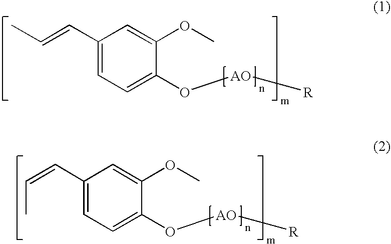
in which
A is C₂- to C₄-alkylene,
n is an integer from 4 to 900,
m is an integer from 1 to 50, and
R is hydrogen, an m-valent acid group, or an m-valent organic radical having 1 to 200 carbon atoms which, apart from carbon and hydrogen, can also contain a heteroatom selected from the group consisting of oxygen, nitrogen, sulphur, phosphorus, and mixtures thereof.

2. A compound as claimed in claim 1, in which A is an ethylene radical or a mixture of ethylene and propylene radicals.

3. A compound as claimed in claim 1, in which m is an integer from 1 to 10.

4. (canceled)

5. A process for the preparation of an emulsified copolymer, said process comprising:
a) mixing a compound of the formula 1 or 2 or a mixture thereof 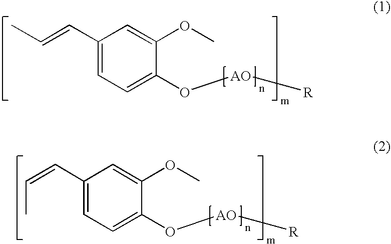
in which
A is C₂- to C₄-alkylene,
n is an integer from 4 to 900,
m is an integer from 1 to 50, and
R is hydrogen, an m-valent acid group, or an m-valent organic radical having 1 to 200 carbon atoms which, apart from carbon and hydrogen, can also contain a heteroatom selected from the group consisting of oxygen, nitrogen, sulphur, phosphorus, and mixtures thereof,
with at least one olefinically unsaturated monomer capable of free-radical polymerization, and
b) polymerizing the resulting mixture in aqueous phase to provide the emulsified copolymer.

6. (canceled)

7. The process of claim 5, where the olefinically unsaturated monomer is selected from the group consisting of open-chain N-vinylamide, cyclic N-vinylamide (N-vinyl lactam) with a ring size of from 3 to 9, a amide of acrylic acid, an amide of methacrylic acid, alkoxylated acrylamide, alkoxylated methacrylamide, N,N-dimethylaminomethacrylate; diethylaminomethyl methacrylate, acrylamidoglycolic acid, methacrylamidoglycolic acid, 2- and 4-vinylpyridine, vinyl acetate, glycidyl methacrylate, styrene, acrylonitrile, diallylamine, diallyldimethylammonium chloride, trimethyl-2-methacryloylethylammonium chloride, ethylene, propene, butane, pentene, 1,3-butadiene and chloroprene, vinyl chloride, vinylidene chloride, vinylidene fluoride, an alkoxylated allyl compound, a vinyl compound, vinyl ether, olefinically unsaturated carboxylic acid ester, acrylic acid, methacrylic acid, styrenesulfonic acid, maleic acid, fumaric acid, crotonic acid, itaconic acid, senecioic acid, acrylamidomethylpropanesulfonic acid, methallylsulfonic acid, a vinylphosphonic acid derivative, and mixtures thereof.

8. An emulsified copolymer in an aqueous phase prepared by the process of claim 5.

9. An aqueous dispersion comprising the emulsified copolymer prepared by the process of claim 5.
